(19) United States
(12) Patent Application Publication
(10) Pub. No.: US 2008/0077094 A1
(43) Pub. Date: Mar. 27, 2008

(54) DOSE SETTING MECHANISM AND METHOD

(76) Inventors: Stefan Burren, Bremgarten (CH); Ulrich Moser, Heimiswil (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/842,664

(22) Filed: Aug. 21, 2007

Related U.S. Application Data
(63) Continuation of application No. PCT/CH2006/000019, filed on Jan. 11, 2006.

(30) Foreign Application Priority Data
Feb. 22, 2005 (DE) 10 2005 008 065.0

(51) Int. Cl. A61M 5/315

(52) U.S. Cl. 604/209

(57) ABSTRACT

An injection device for a dosed administration of an injectable substance and a method of the preparation for the dosed administration, wherein the device includes a toothed rack with a longitudinal length, locking members associated with the rack and locking elements, wherein adjacent locking members have a first separation from each other and the locking elements have a second separation from each other, the first separation being different from the second separation, the method including making a priming movement and a dispensing movement.

### CROSS-REFERENCE TO RELATED APPLICATIONS

This application is a continuation of International Application No. PCT/CH2006/000019 filed on Jan. 11, 2006, which claims priority to German. Application No. DE 10 2005 008 065.0 filed on Feb. 22, 2005, the contents of both of which are incorporated in their entirety herein by reference.

### BACKGROUND

The present invention relates to devices and methods for injecting, infusing, administering, dispensing or delivering a substance, and to methods of making and using such devices. Most particularly, the present invention relates to injection devices for administering set doses of an injectable product, wherein the injection devices include a dose setting mechanism. More particularly, it relates to an injection device such as those used in the medical field, with a toothed rack and a plurality of locking members and locking elements, whereby fine doses of an injectable product which usually require toothed racks with smaller distances between the locking elements can be set by the dose setting mechanism.

Patent specification EP 1 228 777 B1 describes an injection unit by which several injections can be administered, in which locking elements of a toothed rack are offset from one another on oppositely lying sides of the toothed rack along the longitudinal length of the rack, thereby making it easier to set a finer dose quantity of an injectable product.

### SUMMARY

One object of the present invention is to provide a device and a method of administering set doses of an injectable product from an injection device by which smaller dose quantities of an injectable product can be set and administered.

In one embodiment the present invention comprises an injection device, a dose setting mechanism for the injection device and a method of setting or selecting a dose to be injected, wherein the dose setting mechanism comprises a tooth meshing system with an uneven pitch.

In one embodiment, the present invention comprises an injection device for administering selected doses of an injectable substance, the injection device comprising a dose selecting mechanism comprising a rack having a longitudinal length and carrying a plurality of teeth along the length, said teeth at a first distance from each other, and at least two locking elements along the length of the rack at a second distance from each other, wherein the first distance is different than the second distance.

In one embodiment, the present invention comprises an injection device for a dosed administration of an injectable substance and a method of the preparation for the dosed administration, wherein the device includes a toothed rack with a longitudinal length, locking members associated with the rack and locking elements, wherein adjacent locking members have a first separation from each other and the locking elements have a second separation from each other, the first separation being different from the second separation, the method including making a priming movement and a dispensing movement.

In one embodiment, the dose setting mechanism of the present invention for administering an injectable product from an injection device comprises a toothed rack (which also might be thought of and referred to as a generally elongated rod or rod-like element) with a plurality of locking members, e.g., teeth, texturing, serrations or cams of a sawtooth design (e.g., a pattern, series or sequence of alternating high and low points or areas, peaks and dips or valleys, raised and relieved areas or regions, etc.). The locking members are disposed along the longitudinal axis of the toothed rack and are disposed respectively at a distance from one another which may be constant from locking member to locking member or which may be different. In one embodiment, the device of the present invention also comprises at least two locking elements, which may be disposed on a guide piece and/or on a driving sleeve or on a snapper or catch element of a guide piece or a driving sleeve, and which are disposed at a distance from one another along the longitudinal axis or length of the toothed rack of the injection device.

In the situation where more than two locking elements are disposed on or associated with the injection device, the respective distances between the locking elements may be constant or different along the longitudinal length of the rack. For the purpose of the invention, the first constant distance is between the locking members of the toothed rack or the first distances are between the locking members of the toothed rack, and the respective distances between the locking members may be different, not the same as the second constant distance between the locking elements or the second distances between the locking elements, and the respective distances between the locking elements may be different.

In some preferred embodiments, the first distance or the respective first distances between the locking members or teeth of the rack may be smaller or larger than the second distance or the second respective distances between the locking elements, in which case the distances are selected by reference to distances in the longitudinal direction or along the length of the toothed rack or injection device.

In some embodiments, it may be preferable for the locking elements, or catch elements on which the locking elements may be disposed, to be disposed at an identical position relative to the circumference of the toothed rack, and to be offset from one another or separated from one another longitudinally relative to the rack by a second constant distance or by non-constant second distances. Thus, locking elements can be provided on only one side of the toothed rack, for example, which are able to engage in the locking members, i.e., the teeth, of the toothed rack. The locking elements may also be disposed offset from one another or spaced apart from one another circumferentially, in which case locking elements may lie opposite one another. In one embodiment, the locking elements may be disposed at an identical position and/or offset from one another longitudinally so that locking elements may lie opposite one another. In another embodiment, they may be disposed in an identical position longitudinally or may be offset from one another circumferentially and longitudinally.

In some embodiments, two, three, four, five, six, seven, eight or more than eight locking elements may be provided on the injection device, and an equal number of locking elements are disposed on the injection device, in some embodiments, in an identical position in the circumferential direction and one after the other in the longitudinal direction and with a same number of locking elements in turn disposed lying opposite them in an identical position in the longitudinal direction.

In some embodiments, the locking elements in the longitudinal direction of the toothed rack may be disposed at a constant distance from one another, in which case not every one of the locking elements latches in the locking members of the toothed rack and instead, for example, every second locking element in the longitudinal direction of the toothed rack latches in the locking members of the toothed rack. In this respect, the terms latching, locking, lodging, meshing and the like should be construed as meaning that the locking elements lie with a contact side against a co-operating stop of a locking member. In other words, the side of the locking elements or cams extends or stands perpendicular to the toothed rack in the state in which the locking elements lie between the locking members of the toothed rack, and the co-operating stop constitutes the side of the locking members or teeth of the rack which extend or stand perpendicular on the rack. The other locking elements do not latch into the locking members of the toothed rack and therefore do not lie with the contact side against a co-operating stop of a locking member. Due to the different distance between the locking members of the rack compared with the distance between the locking elements, which may be both constant and non-constant from one locking element to the next, not every contact side of a locking element but only every second, third, fourth, fifth or sixth contact side of a locking element, for example, lies against a co-operating stop of a locking member, and the remaining or other locking elements do not latch with the locking members of the rack, in other words do not lie with their contact sides against a co-operating stop of a locking member.

If, for example, every n-th locking element latches in the teeth of the rack, in other words every n-th locking element lies with its contact side against a co-operating stop of a locking member of the rack, the other or remaining locking elements do not latch in the rack and do not lie with their contact side lying against a co-operating stop of a locking member of the rack. The respective distances needed between the locking elements needed for this can be calculated as a function of the distance between the locking members of the rack.

If the first distance between the locking members of the rack is shorter than the second distance between the locking elements, the respective distance between the locking elements may be represented as a function of the distance between the locking members of the rack as follows:

$$A 2 = A 1. \frac{n \cdot k + 1}{n} ; k ɛ {N}^{⋀} n ɛ N$$

This being the case, the value k, which may be selected from any of the natural numbers, may be varied for every calculation of a distance of the locking elements from one another and, if the value of k remains the same, constant distances will result between the consecutive locking elements. The value n, which may be selected from any of the natural numbers, is used to determine which of every so many locking elements will lie with its contact side against a co-operating stop of the toothed rack to transmit force to the toothed rack, to move the toothed rack or to prevent a movement of the toothed rack. If the value range of k is broadened to k ε N o, this will enable allowance to be made for the special situation where if k=O, the distances between the locking elements will be minimal and will depend solely on the value of n.

If the first distance between the locking members of the toothed rack is larger than the second distance between the locking elements, the distance between the locking elements as a function of the distance between the locking members of the rack will be as follows:

$$A 2 = A 1. \frac{n \cdot k - 1}{n} ; k ɛ {N}^{⋀} n ɛ N$$

In some embodiments, the locking elements of an injection device in accordance with the present invention are non-elastic and are provided in the form of generally sawtooth cams or teeth. The cams or teeth have a contact side and a sliding side, in which case the contact side refers to the side of the cam which is generally perpendicular to the toothed rack when the locking elements are disposed between the locking members of the toothed rack. In this position, the sliding side refers to the side of the cam or locking element disposed obliquely on the rack.

In some embodiments, the locking elements may be moved radially relative to the rack or may be provided in the form of a catch element or a snapper, which can be moved radially relative to the rack. The locking elements may also be provided on a driving sleeve which can be moved relative to the rack or on a guide piece, in which case the rack can be moved relative to the guide piece. The driving sleeve and the guide piece on which the locking elements may be disposed may be moved radially, for example, relative to the toothed rack.

In some embodiments, the locking members or teeth of the toothed rack may be disposed across the entire length of the rack and may be disposed at constant distances from one another along the longitudinal axis or length of the rack, for example, or they may be disposed intermittently at constant distances from one another, in which case the locking members in a first portion along the longitudinal length of the rack are respectively disposed at a first constant distance from one another and, in a second portion following, adjoining or adjacent to the first portion, may be respectively disposed at a second constant distance from one another, in which case the first distance between the locking members in the first portion and the second distance between the locking members in the second portion may be different. It would also be possible to provide more than two portions following one another with different distances between the locking members, or portions wherein different distances between the locking members may be alternated or periodically alternated, for example.

In some embodiments, the present invention comprises a method of preparing to administer set doses of an injectable product from an injection device. In some embodiments, the method comprises making a first priming movement of the injection device effected by moving a setting device, structure or knob in a direction opposite the direction in which the injectable product is dispensed. The movement of the setting knob causes a driving sleeve to be moved relative to a toothed rack in the direction opposite that in which the injectable product is dispensed. Since at least one locking element of a guide piece latches with locking members of the toothed rack due to a contact side of the at least one locking element of the guide piece lying against a co-operating stop of a locking member of the rack, the rack is held stationary by the guide piece. Accordingly, the contact side of at least one locking element of the guide piece does not lie against a co-operating stop of a locking member of the rack, in other words does not latch with the teeth of the rack. When the priming movement is terminated, a contact side of at least one locking element of the driving sleeve lies against a co-operating stop of a locking member of the rack, and due to the movement of the setting knob in the dispensing direction of the injectable product, the driving sleeve is moved in the dispensing direction and a force is transmitted to a locking member of the rack via the contact side or contact sides lying against a co-operating stop of a locking member of the rack, causing the rack to be moved in the dispensing direction relative to the guide piece. This being the case, at least one contact side of at least one locking element of the driving sleeve does not lie on a co-operating stop of a locking member of the rack. A first dose of the injectable product is then dispensed by moving the rack and, after the relative movement between the rack and guide piece, a contact side of at least one locking element of the guide piece lies against a co-operating stop of a locking member of the rack and a contact side of at least one locking element of the guide piece does not lie against a co-operating stop of a locking member of the rack. A new priming operation can then be performed, followed by an operation to dispense the injectable product.

In some embodiments of the method, every n-th locking element may lie with its contact side against a co-operating stop of a locking member of the rack and transmit a force to the rack to, for example, move the rack, or may absorb a force from the rack to, for example, prevent the rack from moving. In this case, the other or remaining locking elements are not latched with the rack, in other words do not lie with their contact sides against a co-operating stop of a locking member of the rack. After priming the injection device in the direction opposite that in which the injectable product is dispensed and following the movement of the driving sleeve relative to the rack associated with priming, every (n+1)-th locking element of the driving sleeve may lie against a co-operating stop of a locking member of the rack, and the other locking elements of the driving sleeve do not lie with their contact sides on a co-operating stop of a locking member of the rack.

In some embodiments, prior to dispensing the injectable product, every n-th locking element of the guide piece may lie with its contact side against a co-operating stop of a locking member of the rack, for example, in which case the other locking elements of the guide piece do not lie with their contact side on a co-operating stop and, after dispensing the injectable product by moving the rack relative to the guide piece, every (n+1)th locking element of the guide piece latches with the locking members of the rack and the other or remaining locking elements of the guide piece are not latched with the rack.

In some embodiments, after every priming operation, the setting element or knob is moved in the dispensing direction of the injectable product so that a constant quantity of the injectable product is dispensed.

### BRIEF DESCRIPTION OF THE DRAWINGS

FIG. 1 shows one exemplary embodiment of the present invention with two locking elements on oppositely lying arms of a guide piece and driving sleeve, respectively, wherein a distance between locking members of a toothed rack is shorter than the distance between the locking elements;

FIG. 2 shows the injection device illustrated in FIG. 1 with 4 engaging elements on the two oppositely lying arms of the guide piece and driving sleeve respectively;

FIG. 3a is a diagram showing the injection device illustrated in FIG. 2;

FIG. 3b shows the injection device from FIG. 3a prior to priming the injection device and prior to dispensing an injectable product;

FIG. 3c shows the injection device from FIGS. 3a and 3b after dispensing an injectable product;

FIG. 4 illustrates another embodiment of the present invention with three engaging elements on the two oppositely lying arms of the guide piece and driving sleeve, respectively, wherein a distance between the locking members of the rack is larger than the distance between the locking elements;

FIGS. 5a-5c are schematic diagrams of the toothed rack and the locking elements with different distances between the locking elements in the situation where n=2.

### DETAILED DESCRIPTION OF THE DRAWINGS

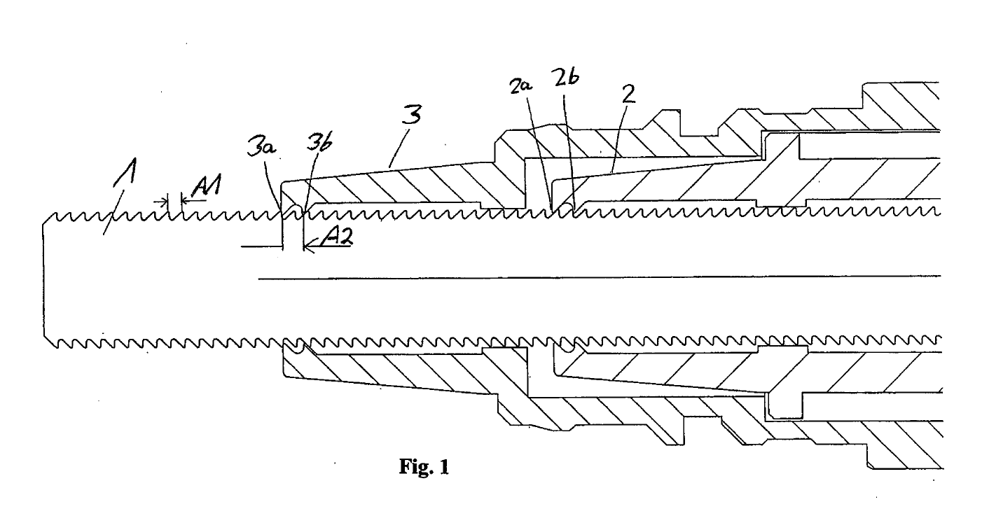

FIG. 1 illustrates one embodiment of the present invention comprising a toothed rack 1, a driving sleeve 2 displaceable relative to the toothed rack 1 and a guide piece 3, wherein the rack 1 can be moved relative to the guide piece 3. Disposed on the two oppositely lying arms of the driving sleeve 2 and on the two oppositely lying arms of the guide piece 3, respectively, are two locking elements 2a, 2b, 3a, 3b, and the distance A2 between the locking elements 2a, 2b, 3a, 3c is larger than the distance A1 between the locking members of the rack 1. The contact side of the second or rear locking element 2b of the driving sleeve 2 in the dispensing direction lies against a co-operating stop of a locking member of the rack 1, so that when the driving sleeve 2 is moved forward, for example, a force can be transmitted to the rack 1 in the dispensing direction by the rear locking element 2b, which lies with its contact side on a co-operating stop of a locking member of the rack, for example, to move the rack 1 in the dispensing direction. In this embodiment, the driving sleeve 2 and hence, the rack 1 can be moved in the dispensing direction until the driving sleeve 2 lies with its stop on the guide piece 3. A dose of injectable product can be set by moving the driving sleeve 2 in the direction opposite the dispensing direction, for example, while the rack remains stationary because it is retained by the guide piece 3. The rack 1 is retained by the front locking element 3a of the guide piece 3, which lies with its contact side on a co-operating stop of a locking member of the rack 1, and the rear locking element 3b of the guide piece 3 does not lie with its contact side against a co-operating stop of a locking member of the rack 1. This embodiment of the present invention enables very fine doses to be set by moving the driving sleeve 2 in the direction opposite the dispensing direction and a priming movement is effected whereby, when the driving sleeve 2 has been moved by one locking member or one click (in some embodiments, the relative movement may generate a clicking sound) further in the direction opposite the dispensing direction, it is no longer the rear 2b but the front locking element 2a of the driving sleeve 2 which lies with its contact side against a co-operating stop of a locking member of the rack 1 and a force is transmitted from the driving sleeve 2 to the toothed rack 1 in the dispensing direction to run or perform a dispensing operation.

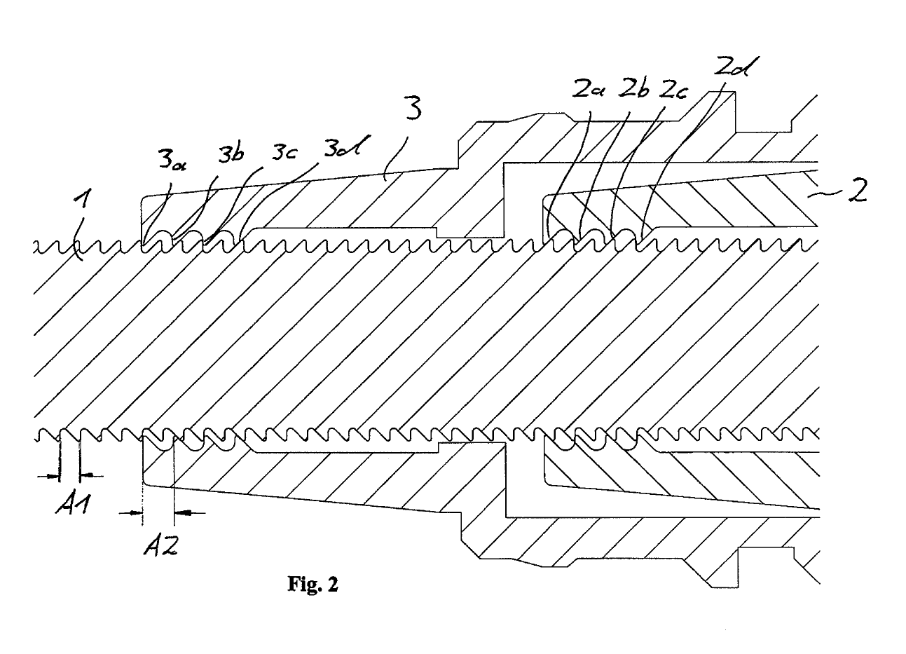

FIG. 2 illustrates a different embodiment of the injection device illustrated in FIG. 1, in which four locking elements 2a, 2b, 2c, 2d, 3a, 3b, 3c, 3d are provided on each arm of the driving sleeve 2 and the guide piece 3. Every second locking element of the guide piece 3 and the driving sleeve 2 engages in the locking members or teeth of the rack 1 and every second locking element of the guide piece 3 and the driving sleeve 2 does not engage with the rack 1. Accordingly, the second and fourth locking elements 2b, 2d of the driving sleeve 2 lie with their contact side against a co-operating stop of a locking member to enable a force to be transmitted to the rack 1 or to move the rack 1, and every first 3a and third locking element 3c of the guide piece 3 lies with its contact side against a co-operating stop of a locking member of the rack 1 to enable a force from the rack 1 to be absorbed or to prevent the rack 1 from moving. The same dose quantities can be set with the injection device illustrated in FIG. 2 as with the injection device illustrated in FIG. 1 and, in this case, because there are four locking elements on the arms of the driving sleeve 2 and the guide piece 3, a very strong rack 1 is achieved along with a precise or exact movement of the rack 1.

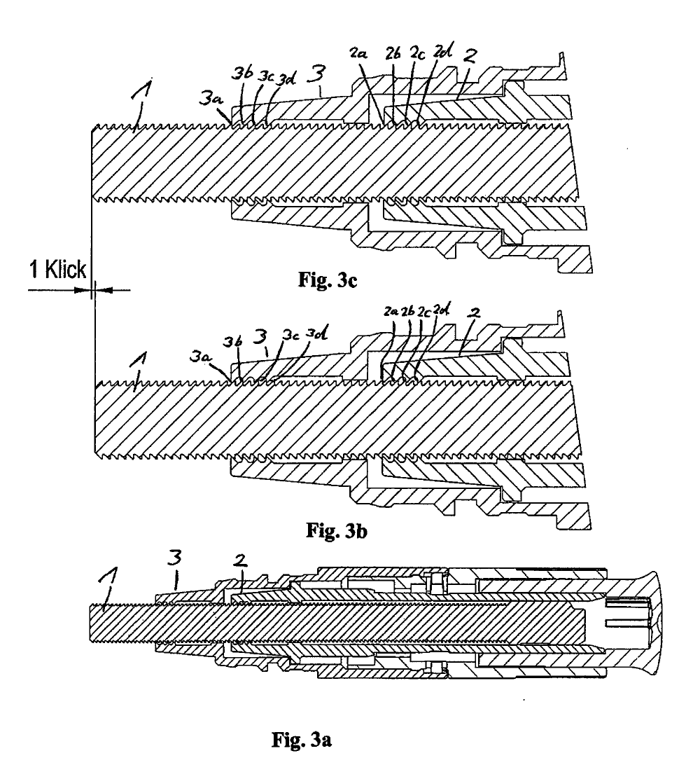

FIG. 3a shows an overall view of the injection device illustrated in FIG. 2 while FIGS. 3b and 3c illustrate the injection device in two different modes. FIG. 3a shows the injection device in a position prior to a priming and dispensing operation, in which the first 2a and third locking element 2c of the driving sleeve 2 lie against a locking member of the rack 1 or latch with the rack 1. Accordingly, a movement for priming the injection device can be effected by moving a setting element, e.g., knob, in the direction opposite the dispensing direction. The driving sleeve 2 is moved further in the direction opposite the dispensing direction by one locking member or one click so that it is now the second 2b and fourth locking elements 2c of the driving sleeve 2 which lie against a locking member of the rack 1, as illustrated in FIG. 3b, because the rack 1 remains stationary and the driving sleeve 2 therefore effects a relative movement with respect to the rack 1. As the driving sleeve 2 is moved in the direction opposite the dispensing direction, the rack 1 is held stationary by the guide piece 3, in particular by the second 3b and fourth locking element 3d of the guide piece 3, as illustrated in FIG. 3a, because the second 3b and fourth locking element 3d lie with their contact side against a co-operating stop of locking members and thus prevent any movement of the rack 1. Once the injection device has been primed, the injectable product can be dispensed by moving the setting element and hence, the driving sleeve 2 in the dispensing direction so that the second 2b and the fourth locking element 2d of the driving sleeve 2 transmit a force to the rack 1 in the dispensing direction. Thus, the rack 1 moves in the dispensing direction and an injectable product is dispensed as the rack 1 is moved relative to the guide piece 3. When the rack 1 is moved in the dispensing direction by one locking member or one click relative to the guide piece 3, the first 3a and third locking element 3c of the guide piece 3 lie against locking members of the rack 1. A small dose is dispensed due to the movement by one click, i.e., the rack 1 covers a shorter distance in the dispensing direction than the distance between locking members or between two locking elements.

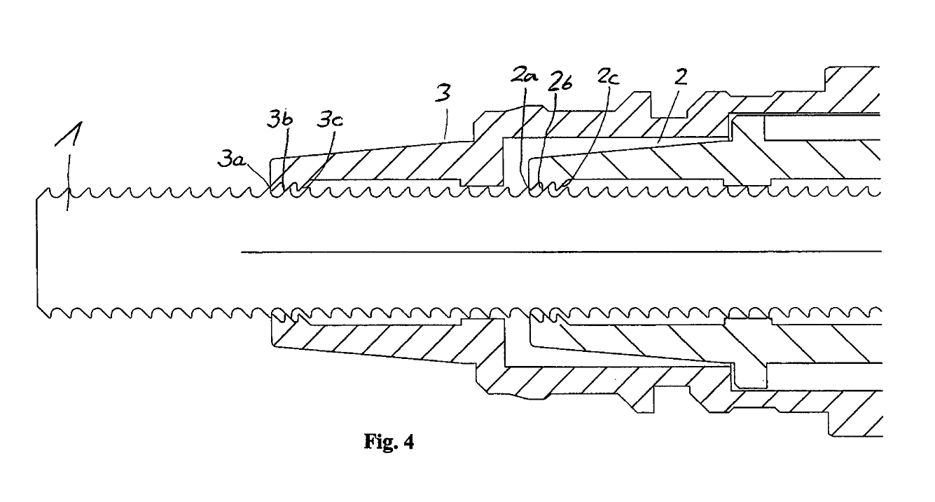

FIG. 4 shows another embodiment of the present invention, in which, in contrast with the injection device illustrated in FIG. 1, the distances between the locking members of the rack 1 are larger than the distances between the locking elements 2a, 2b, 3c, 3a, 3b, 3c. Accordingly, only the first locking element 2a, 3a of the guide piece 3 and driving sleeve 2 lie respectively on a co-operating stop of a locking member or tooth of the rack 1, while the other locking elements 2b, 2c, 3b, 3c are not latched with the rack 1 and therefore have no active connection to the rack 1 to enable a force to be transmitted to the rack 1. If the driving sleeve 2 is moved relative to the rack 1 in the priming direction, for example, so that the rack 1 is held stationary by the first locking element 3a of the guide piece 3, it is no longer the first locking element 2a of the driving sleeve which is actively connected to the rack 1 but the second locking element 2b of the driving sleeve 2, which lies with its contact side against a co-operating stop of a locking member of the rack 1 and establishes an active connection to the rack 1. If the driving sleeve 2 is now moved in the dispensing direction, for example, the second tooth 2b of the driving sleeve 2 transmits a force to the rack 1 and moves it relative to the guide piece 3 further by one click or one locking member. After this movement, the second locking element 3b of the guide piece 3 lies against a locking member of the rack 1. Due to the movement of the rack 1 relative to the guide piece 3, an injectable product, such as a liquid, can be dispensed in fine doses.

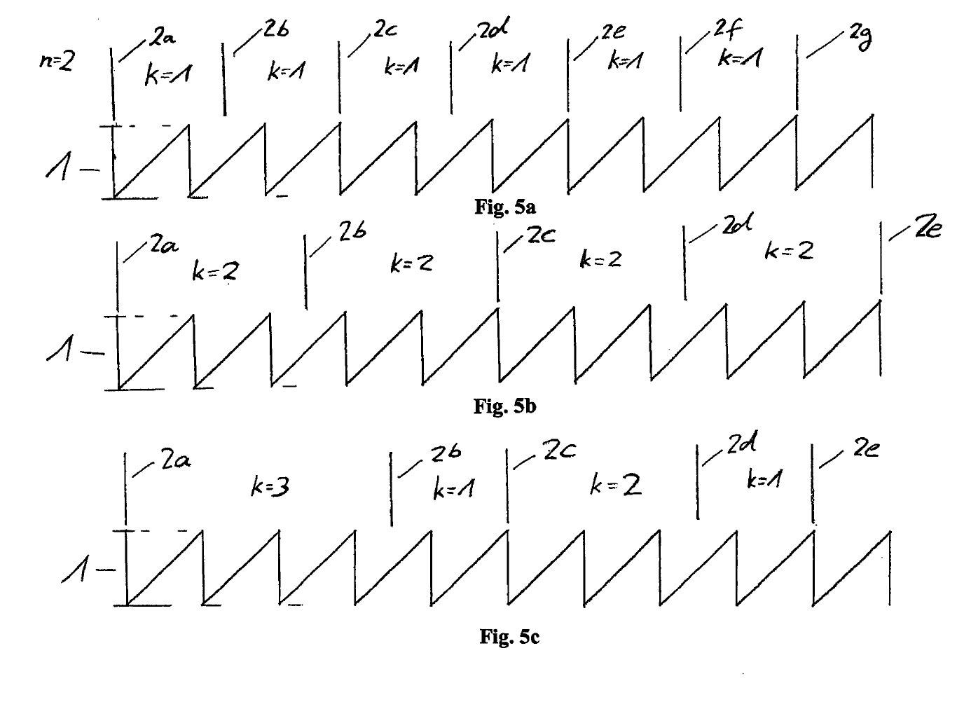

FIGS. 5a, 5b and 5c are schematic diagrams illustrating the rack 1 and the locking elements for the situation where n=2, in other words the situation in which every second locking element lies with its contact side against a co-operating stop of a locking member of the rack 1. In this instance, the distance between the locking elements is larger than the distance between the locking members.

In FIG. 5a, to calculate each distance, the free value k is selected as 1 so that the distances between the locking elements are 1½ times the distance between the locking members, in other words the distance between the locking elements is 3/2-times the distance between the locking members. The same function demonstrated in FIG. 5a and the same characteristic wherein every second locking element lies against a locking member is also achieved if the free parameter k is selected as 2. In this case, as illustrated in FIG. 5b, the distance between the locking elements is a distance 5/2-times the distance between the locking members of the rack 1. Here too, a dose quantity can be achieved which corresponds to half the distance between the locking members. As illustrated in FIG. 5c, the distance between the locking elements need not be constant but conforms to a formula as follows:

$$A 2 = A 1. \frac{n \cdot k + 1}{n}$$

whereby the dose quantities which can be set correspond to a half distance between the locking members, for example. In FIG. 5c, therefore, the distance between the first and the second locking element based on a value of k=3 is 7/2-times the distance between the locking members of the rack 1, while the distance between the second and the third locking element corresponds to 3/2-times, the distance between the third and fourth locking element corresponds to 5/2-times and the distance between the fourth and fifth locking element again corresponds to 3/2-times the distance between the locking members. It is also possible to select any other whole-number values for k to achieve the same function and/or dose setting system.

Embodiments of the present invention, including preferred embodiments, have been presented for the purpose of illustration and description. They are not intended to be exhaustive or to limit the invention to the precise forms and steps disclosed. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A dose setting mechanism for administering set doses of an injectable product from an injection device, comprising:
a toothed rack with a plurality of locking members, which locking members are disposed along a longitudinal length of the toothed rack at a first distance from one another; and
at least two locking elements, which locking elements are disposed along the longitudinal length of the toothed rack at a second distance from one another;
wherein
the first distance is different than the second distance.

2. The dose setting mechanism as claimed in claim 1, wherein the first distance is shorter than the second distance.

3. The dose setting mechanism as claimed in claim 1, wherein the first distance is larger than the second distance.

4. The dose setting mechanism as claimed in claim 1, wherein the locking elements are at a fixed ratio in terms of their position relative to one another.

5. The dose setting mechanism as claimed in claim 1, wherein the locking elements are in an identical position circumferentially relative to the rack.

6. The dose setting mechanism as claimed in claim 1, wherein the locking elements are at least one of in an identical position circumferentially related to the rack and offset from one another.

7. The dose setting mechanism as claimed in claim 1, wherein the locking elements lie opposite one another.

8. The dose setting mechanism as claimed in claim 1, wherein the locking elements are offset from one another circumferentially relative to the rack and are in an identical position along the longitudinal length of the rack.

9. The dose setting mechanism as claimed in claim 1, wherein the locking elements are in an identical position circumferentially relative to the rack and are offset from one another along the longitudinal length of the rack.

10. The dose setting mechanism as claimed in claim 1, wherein the locking elements are at a constant distance from one another along the longitudinal length of the rack.

11. The dose setting mechanism as claimed in claim 1, comprising two, three, four, five, six, seven, eight or more locking elements.

12. The dose setting mechanism as claimed in claim 9, wherein the locking elements are at a constant distance from one another along the longitudinal length of the rack and each comprises a contact side, the contact side of every second, third, fourth, fifth, or sixth locking element lying against a co-operating stop of a locking member along the longitudinal length of the rack and the contact side of the other locking elements not lying against a co-operating stop of a locking member of the rack.

13. The dose setting mechanism as claimed in claim 1, wherein each of the locking elements comprises a contact side and each of the locking members comprises a stop, and wherein every n-th locking element lies with its contact side against a co-operating stop of a locking member and the other locking elements do not lie with their contact side against a co-operating stop of a locking member, the first distance being shorter than the second distance, and the second distance is a function of the first distance, wherein:
$$A 2 = \frac{n \cdot k + 1}{n} A 1$$
and wherein k and n may be selected from any of the natural numbers.

14. The dose setting mechanism as claimed in claim 1, wherein each of the locking elements comprises a contact side and each of the locking members comprises a stop, and wherein every n-th locking element lies with its contact side against a co-operating stop of a locking member, the other locking elements do not lie with their contact side against a co-operating stop of a locking member, the first distance being larger than the second distance, and the second distance is described as a function of the first distance, wherein:
$$A 2 = \frac{n \cdot k - 1}{n} A 1$$
and wherein k and n may be selected from any of the natural numbers.

15. The dose setting mechanism as claimed in claim 1, wherein the locking elements are non-elastic sawtooth cams comprising one perpendicular contact side and one oblique sliding side.

16. The dose setting mechanism as claimed in claim 1, wherein the locking elements are moveable radially relative to the rack.

17. The dose setting mechanism as claimed in claim 1, wherein the locking elements are on a driving sleeve moveable relative to the rack and on a guide piece, wherein the rack is moveable relative to one of the guide piece.

18. The dose setting mechanism as claimed in claim 1, wherein the locking members of are spaced at constant distances along the length of the rack and along the longitudinal axis of the rack.

19. The dose setting mechanism as claimed claim 1, wherein the locking members are respectively at a constant distance from one another in a first portion along the longitudinal length of the rack and are at a second constant distance from one another in an adjacent second portion along the longitudinal length of the rack, wherein the first distance and the second distance are different.

20. A method of preparing to administer set doses of an injectable product from an injection device comprising a setting element, a driving sleeve, a toothed rack comprising locking members, and a guide piece comprising locking elements, the method comprising the steps of:
making a first priming movement of the injection device by moving the setting element in a direction opposite a direction in which the injectable product is dispensed, the movement of the setting element causing the driving sleeve to be moved in the direction opposite the direction in which the injectable product is dispensed relative to the toothed rack, wherein, because a contact side of at least one locking element of the guide piece lies against a co-operating stop of a locking member of the toothed rack, the toothed rack remains stationary, and the contact side of at least one locking element of the guide piece does not lie against a co-operating stop of a locking member of the toothed rack, wherein when the priming movement is terminated, at least one contact side of at least one locking element of the driving sleeve lies against a co-operating stop of a locking member of the toothed rack so that due to the movement of the setting element in the direction in which the injectable product is dispensed, the driving sleeve moves the toothed rack in the dispensing direction relative to the guide piece and a contact side of at least one locking element of the driving sleeve does not lie against a co-operating stop of a locking member of the toothed rack; and
dispensing a first dose due to the movement of the toothed rack and after the relative movement between the toothed rack and guide piece, a contact side of at least one locking element of the guide piece lies against a co-operating stop of a locking member of the toothed rack and a contact side of at least one locking element of the guide piece does not lie against a co-operating stop of a locking member of the toothed rack.

21. The method of preparing to administer set doses of an injectable product from an injection device as claimed in claim 20, wherein in the initial position, every n-th locking element of the driving sleeve lies with its contact side against a co-operating stop of a locking member of the toothed rack, and the other locking elements of the driving sleeve do not lie with their contact side against a co-operating stop of a locking member of the toothed rack, and after priming in the direction opposite that in which the injectable product is dispensed, every (n+1)-th locking element of the driving sleeve lies with its contact side against a co-operating stop of a locking member of the toothed rack and the other locking elements of the driving sleeve do not lie with their contact side against a co-operating stop of a locking member of the toothed rack.

22. The method of preparing to administer set doses of an injectable product from an injection device as claimed in claim 20, wherein after a priming movement of the injection device, every n-th locking element of the guide piece lies with its contact side against a co-operating stop of a locking member of the toothed rack and the other locking elements of the guide piece do not lie with their contact side against a co-operating stop of a locking member of the toothed rack, the toothed rack being moved relative to the guide piece during the dispensing operation and after the dispensing operation, every (n+1)-th locking element of the guide piece lies with its contact side against a co-operating stop of a locking member of the toothed rack and the other locking elements of the guide piece do not lie with their contact side against a co-operating stop of a locking member of the toothed rack.

23. The method for preparing to administer set doses of an injectable product from an injection device as claimed in claim 20, wherein after every priming movement, constant dose quantities of the injectable product can be dispensed by moving the setting element in the direction in which the injectable product is dispensed.

24. An injection device for administering selected doses of an injectable substance, the injection device comprising a dose selecting mechanism comprising a rack having a longitudinal length and carrying a plurality of teeth along the length, said teeth at a first distance from each other, and at least two locking elements along the length of the rack at a second distance from each other, wherein the first distance is different than the second distance.

25. The injection device according to claim 24, wherein each of the locking elements comprises a contact side and each tooth of the teeth comprises a stop, and wherein every n-th locking element lies with its contact side against a co-operating stop of a tooth, the other locking elements do not lie with their contact side against a co-operating stop of tooth, the first distance being larger than the second distance, and the second distance is described as a function of the first distance, wherein:
$$A 2 = \frac{n \cdot k + 1}{n} A 1$$
and wherein k and n may be selected from any of the natural numbers.
